(19) United States
(12) Patent Application Publication
(10) Pub. No.: US 2016/0249796 A1
(43) Pub. Date: Sep. 1, 2016

(54) TREATMENT METHOD

(71) Applicant: OLYMPUS CORPORATION, Tokyo (JP)

(72) Inventor: Ken FUJISAKI, Sagamihara-shi (JP)

(21) Appl. No.: 14/634,168

(22) Filed: Feb. 27, 2015

(51) Int. Cl. A61B 1/233; A61B 1/04; A61B 1/00

(52) U.S. Cl. CPC A61B 1/233; A61B 1/00154; A61B 1/04; A61B 2019/5257

(57) ABSTRACT

A treatment method includes: inserting a treatment member into a maxillary sinus through a first opening in a nasal cavity leading to the maxillary sinus; inserting an endoscope into the maxillary sinus through a second opening leading to the maxillary sinus and different from the first opening; and performing a treatment on a treatment target site in the maxillary sinus while the treatment member and the treatment target site exist within a visual field of the endoscope.

### BACKGROUND OF THE INVENTION

1. Field of the Invention

The present invention relates to a treatment method for treating a treatment target site in a maxillary sinus.

2. Description of the Related Art

Conventionally, there is known a technique for performing a treatment by inserting a medical device for a treatment into a paranasal sinus. FIG. 42 is a schematic view of a paranasal sinus. As illustrated in FIG. 42, the paranasal sinus includes a frontal sinus Fs, an ethmoid sinus Es, a maxillary sinus Ms, and a sphenoid sinus Ss. The paranasal sinus (frontal sinus Fs, ethmoid sinus Es, maxillary sinus Ms, and sphenoid sinus Ss) leads to the inside of a nasal cavity through an opening.

For example, FIG. 43 is a schematic view illustrating a natural ostium V leading to the maxillary sinus Ms in the nasal cavity. The natural ostium V is located on the back side of an uncinate process Up as seen from the nasal cavity. Therefore, a practitioner such as a doctor performs a treatment in the maxillary sinus by inserting a medical device beyond the uncinate process Up while bringing the medical device close to the ostium V.

As a related technique for inserting the medical device into the natural ostium V leading to the maxillary sinus Ms, for example, a technique is known in which an expandable body such as a balloon is arranged at a distal end part of the medical device to enlarge the natural ostium V using the expandable body. According to this related technique, even in a case of stenosis of the natural ostium V caused by an inflammation or the like of the maxillary sinus MS, it is possible to insert the medical device for a treatment tool into the natural ostium V leading to the maxillary sinus MS.

### SUMMARY OF THE INVENTION

A treatment method according to one aspect of the invention includes: inserting a treatment member into a maxillary sinus through a first opening in a nasal cavity leading to the maxillary sinus; inserting an endoscope into the maxillary sinus through a second opening leading to the maxillary sinus and different from the first opening; and performing a treatment on a treatment target site in the maxillary sinus while the treatment member and the treatment target site exist within a visual field of the endoscope.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is an outline illustrating a structure of an endoscope system according to a first embodiment of the present invention;

FIG. 2 is a top view illustrating structures of an endoscope and an auxiliary insertion tool according to the first embodiment of the present invention;

FIG. 3 is a schematic view illustrating a treatment method for a maxillary sinus according to the first embodiment of the present invention;

FIG. 4 is a schematic view illustrating the treatment method for a maxillary sinus according to the first embodiment of the present invention;

FIG. 5 is a schematic view illustrating the treatment method for a maxillary sinus according to the first embodiment of the present invention;

FIG. 6 is a schematic view illustrating the treatment method for a maxillary sinus according to the first embodiment of the present invention;

FIG. 7 is a schematic view illustrating the treatment method for a maxillary sinus according to the first embodiment of the present invention;

FIG. 8 is a schematic view illustrating the treatment method for a maxillary sinus according to the first embodiment of the present invention;

FIG. 9 is a schematic view illustrating the treatment method for a maxillary sinus according to the first embodiment of the present invention;

FIG. 10 is a schematic view illustrating the treatment method for a maxillary sinus according to the first embodiment of the present invention;

FIG. 11 is a schematic view illustrating the treatment method for a maxillary sinus according to the first embodiment of the present invention;

FIG. 12 is a schematic view illustrating the treatment method for a maxillary sinus according to the first embodiment of the present invention;

FIG. 13 is a schematic view illustrating the treatment method for a maxillary sinus according to the first embodiment of the present invention;

FIG. 14 is a schematic view illustrating the treatment method for a maxillary sinus according to the first embodiment of the present invention;

FIG. 15 is a schematic view illustrating the treatment method for a maxillary sinus according to the first embodiment of the present invention;

FIG. 16 is a flowchart illustrating the treatment method for a maxillary sinus according to the first embodiment of the present invention;

FIG. 17 is a schematic view illustrating a treatment method for a maxillary sinus according to a first modified example of the first embodiment of the present invention;

FIG. 18 is a schematic view illustrating the treatment method for a maxillary sinus according to the first modified example of the first embodiment of the present invention;

FIG. 19 is a schematic view illustrating the treatment method for a maxillary sinus according to the first modified example of the first embodiment of the present invention;

FIG. 20 is a schematic view illustrating the treatment method for a maxillary sinus according to the first modified example of the first embodiment of the present invention;

FIG. 21 is a schematic view illustrating a treatment method for a maxillary sinus according to a second modified example of the first embodiment of the present invention, and illustrating an example of a treatment tool to be inserted into a guide sheath;

FIG. 22 is a schematic view illustrating a treatment method for a maxillary sinus according to a third modified example of the first embodiment of the present invention, and illustrating an example of the treatment tool to be inserted into the guide sheath;

FIG. 23 is a schematic view illustrating a treatment method for a maxillary sinus according to a fourth modified example of the first embodiment of the present invention, and illustrating an example of the treatment tool to be inserted into the guide sheath;

FIG. 24 is a schematic view illustrating a treatment method for a maxillary sinus according to a fifth modified example of the first embodiment of the present invention, and illustrating an example of the treatment tool to be inserted into the guide sheath;

FIG. 25 is a schematic view illustrating a treatment method for a maxillary sinus according to a second embodiment of the present invention;

FIG. 26 is a schematic view illustrating the treatment method for a maxillary sinus according to the second embodiment of the present invention;

FIG. 27 is a schematic view illustrating the treatment method for a maxillary sinus according to the second embodiment of the present invention;

FIG. 28 is a schematic view illustrating the treatment method for a maxillary sinus according to the second embodiment of the present invention;

FIG. 29 is a schematic view illustrating the treatment method for a maxillary sinus according to the second embodiment of the present invention;

FIG. 30 is a schematic view illustrating the treatment method for a maxillary sinus according to the second embodiment of the present invention;

FIG. 31 is a schematic view illustrating the treatment method for a maxillary sinus according to the second embodiment of the present invention;

FIG. 32 is a schematic view illustrating the treatment method for a maxillary sinus according to the second embodiment of the present invention;

FIG. 33 is a schematic view illustrating the treatment method for a maxillary sinus according to the second embodiment of the present invention;

FIG. 34 is a schematic view illustrating a treatment method for a maxillary sinus according to a first modified example of the second embodiment of the present invention;

FIG. 35 is a schematic view illustrating the treatment method for a maxillary sinus according to the first modified example of the second embodiment of the present invention;

FIG. 36 is a schematic view illustrating the treatment method for a maxillary sinus according to the first modified example of the second embodiment of the present invention;

FIG. 37 is a schematic view illustrating the treatment method for a maxillary sinus according to the first modified example of the second embodiment of the present invention;

FIG. 38 is a schematic view illustrating a treatment method for a maxillary sinus according to a second modified example of the second embodiment of the present invention;

FIG. 39 is a schematic view illustrating the treatment method for a maxillary sinus according to the second modified example of the second embodiment of the present invention;

FIG. 40 is a schematic view illustrating the treatment method for a maxillary sinus according to the second modified example of the second embodiment of the present invention;

FIG. 41 is a schematic view illustrating the treatment method for a maxillary sinus according to the second modified example of the second embodiment of the present invention;

FIG. 42 is a schematic view of a paranasal sinus; and

FIG. 43 is a schematic view illustrating an opening leading to a maxillary sinus.

### DETAILED DESCRIPTION OF THE PREFERRED EMBODIMENTS

Hereinafter, modes for carrying out the present invention (hereinafter, referred to as “embodiments”) will be described in detail with reference to the drawings. Note that the present invention is not limited by the following embodiments. In the following description, each drawing roughly illustrates a shape, a size, and a positional relation to such a degree that the present invention can be understood. Therefore, the present invention is not limited only to the shape, the size, and the positional relation illustrated in each drawing.

### First Embodiment

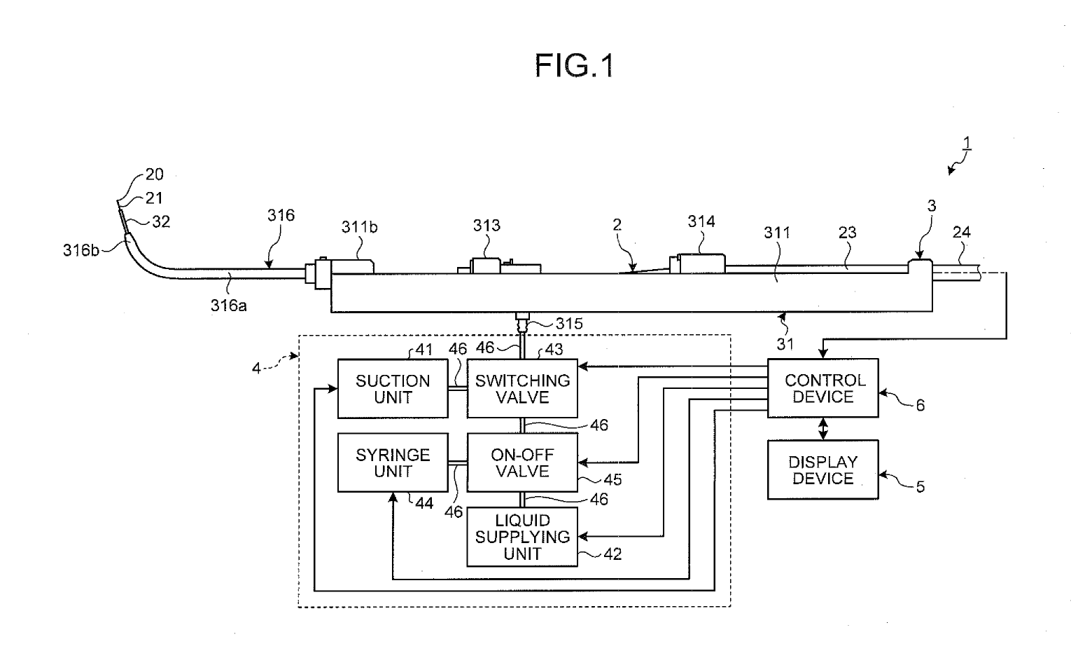

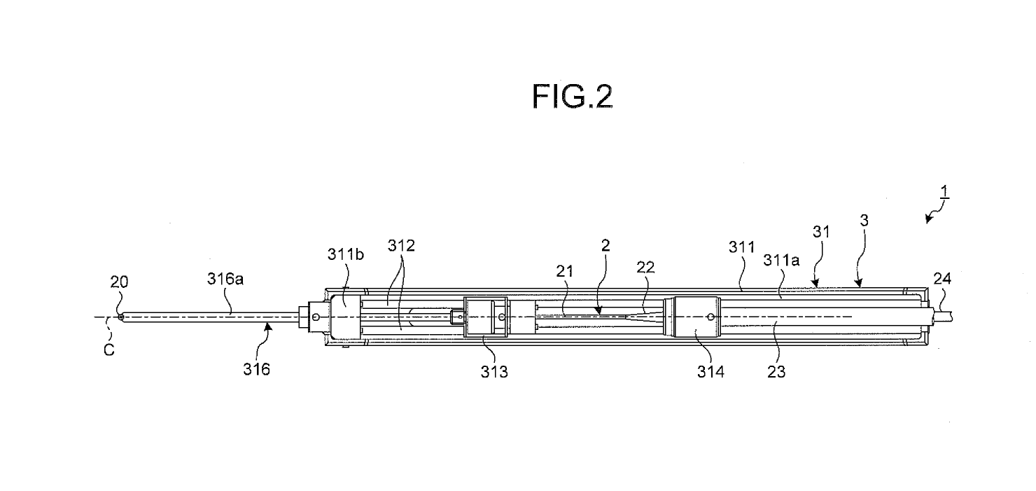

FIG. 1 is an outline illustrating a structure of an endoscope system 1 according to a first embodiment of the present invention. FIG. 2 is a top view illustrating structures of an endoscope 2 and an auxiliary insertion tool 3. The endoscope system 1 illustrated in FIG. 1 includes the scanning endoscope 2, the auxiliary insertion tool 3, a treatment unit 4, a display device 5, and a control device 6 (processor). The endoscope 2 is inserted into a living body of a subject, for example, into a paranasal sinus, and captures an image in the subject to generate an in-vivo image of the subject. The auxiliary insertion tool 3 guides insertion of the endoscope 2 into the subject. The treatment unit 4 supplies liquid, air, or the like for treating the subject, or sucks body fluid or the like of the subject, through the auxiliary insertion tool 3. The display device 5 displays various pieces of information on the endoscope system 1 and an image captured by the endoscope 2. The control device 6 (processor) totally controls elements included in the endoscope system 1.

First, a structure of the endoscope 2 will be described. The endoscope 2 outputs information obtained by imaging the subject to the control device 6. The endoscope 2 includes a distal end part 20, a main body part 21, a bending prevention part 22, a support part 23, and a cable 24. The distal end part 20 includes an optical element (not illustrated) to collect light from a specified visual field. The main body part 21 extends longitudinally from a proximal end of the distal end part 20. The bending prevention part 22 prevents the main body part 21 from bending. The support part 23 supports the endoscope 2 with the auxiliary insertion tool 3. The cable 24 is electrically and optically connected to the control device 6, and transmits an image captured by the endoscope 2 or light. The distal end part 20 and the main body part 21 include an illuminating fiber (not illustrated) to irradiate an object with illumination light through the optical element, and a plurality of light receiving fibers (not illustrated) to receive light reflected by the object through the optical element of the distal end part 20. The distal end part 20 and the main body part 21 are integrally formed so as to be continuous to each other. The distal end part 20 is reinforced by a hard member or the like to prevent the optical element from bending.

Next, a structure of the auxiliary insertion tool 3 will be described. The auxiliary insertion tool 3 includes a handle unit 31 to be held by a practitioner and a guide sheath 32 to guide the endoscope 2 to the paranasal sinus of the subject. In the first embodiment, the guide sheath 32 corresponds to a treatment member.

The handle unit 31 includes a handle main body part 311, a guide rail 312, a first operating unit 313, a second operating unit 314, a joint part 315, and a guide pipe 316.

The handle main body part 311 has a bar shape, and includes a groove part 311a extending in a longitudinal direction. The handle main body part 311 is formed of a rigid material such as a stainless steel material. The handle main body part 311 includes a connecting part 311b, a distal end of which is connected to the guide pipe 316.

The guide rail 312 is arranged in the groove part 311a of the handle main body part 311, and includes a pair of rods, a pair of pipes, or the like disposed in parallel to each other in the longitudinal direction of the handle main body part 311.

The first operating unit 313 supports the guide sheath 32, and is arranged to be movable along the guide rail 312 in the longitudinal direction of the handle main body part 311. The first operating unit 313 moves the guide sheath 32 in a direction of a central axis C of the endoscope 2 and rotates the guide sheath 32 around the central axis C with respect to the guide pipe 316 and the endoscope 2 in response to operation of the practitioner.

The second operating unit 314 supports the support part 23 of the endoscope 2, and is arranged to be movable along the guide rail 312 in the longitudinal direction of the handle main body part 311. The second operating unit 314 moves the endoscope 2 in the direction of the central axis C of the endoscope 2 and rotates the endoscope 2 around the central axis C with respect to the guide pipe 316 and the guide sheath 32 in response to operation of the practitioner.

The joint part 315 protrudes from the handle main body part 311, and is connected to the treatment unit 4 described later through a conduit such as a tube. The joint part 315 connects a conduit (not illustrated) leading to the inside of the guide sheath 32 to the treatment unit 4.

The guide pipe 316 includes a straight pipe part 316a having a cylinder shape and a bent pipe part 316b having a cylinder shape and formed by bending the straight pipe part 316a at a specified angle. The bent pipe part 316b is integrally formed so as to be continued from the straight pipe part 316a. Each of the straight pipe part 316a and the bent pipe part 316b is formed in combination of a steel material such as stainless steel and a flexible member having flexibility, such as silicon resin. Each of the straight pipe part 316a and the bent pipe part 316b has higher rigidity than the guide sheath 32 and the endoscope 2. Each of the straight pipe part 316a and the bent pipe part 316b has such an inner diameter that the guide sheath 32 can be inserted thereinto (can move therein) while the distal end part 20 and the main body part 21 of the endoscope 2 are inserted into (housed in) the guide sheath 32.

A distal end of the guide pipe 316 has a rounded and tapered shape. With this structure, particularly, the guide pipe 316 can be easily inserted into a middle nasal meatus of the nasal cavity, a gap (semilunar hiatus) of an uncinate process, or the like. A distal end of the guide sheath 32 can be arranged near an opening leading to the paranasal sinus.

The guide sheath 32 guides the endoscope 2 to the inside of the paranasal sinus of the subject or to a position adjacent to the opening of the paranasal sinus. The guide sheath 32 houses the endoscope 2 such that the endoscope 2 is movable therein (can be inserted thereinto). At the same time, the guide sheath 32 is inserted into the guide pipe 316, and protrudes from the distal end of the guide pipe 316.

The guide sheath 32 is tubular, and has an inner channel in which the endoscope 2 can move (into which the endoscope 2 can be inserted). The guide sheath 32 is formed using elastically deformable resin or the like. A diameter of an inner periphery of the inner channel is larger than an outer diameter of each of the distal end part 20 and the main body part 21 of the endoscope 2.

Next, a structure of the treatment unit 4 will be described. The treatment unit 4 includes a suction unit 41, a liquid supplying unit 42 (liquid feeding source), a switching valve 43, a syringe unit 44, an on-off valve 45, and a tube 46.

The suction unit 41 is connected to the auxiliary insertion tool 3 through the joint part 315, the tube 46, the switching valve 43, and the on-off valve 45. The suction unit 41 sucks liquid, body fluid, or the like from the subject through the auxiliary insertion tool 3. The suction unit 41 is driven under the control of the control device 6, and thereby sucks a viscous substance existing around an affected part in the paranasal sinus and the nasal cavity, for example. In addition, when the affected part of the subject and a surrounding part thereof are cleaned with physiological saline or the like supplied by the liquid supplying unit 42 described later, the suction unit 41 sucks liquid and a viscous substance (for example, pus) discharged by the cleaning. The suction unit 41 includes a suction pump or the like. As the suction unit 41, an aspirator arranged on the wall or the like of an operating room or a treatment room may be used, for example.

The liquid supplying unit 42 is connected to the auxiliary insertion tool 3 through the joint part 315, the tube 46, and the switching valve 43. The liquid supplying unit 42 supplies liquid into the subject through the auxiliary insertion tool 3. Here, the liquid supplied by the liquid supplying unit 42 is physiological saline or the like. The liquid supplied by the liquid supplying unit 42 is used for cleaning the affected part in the paranasal sinus of the nose, for example.

The switching valve 43 includes a three-way cock or the like. The switching valve 43 is connected to each of the tube 46, the suction unit 41, and the liquid supplying unit 42 through the on-off valve 45. The switching valve 43 selectively switches a part connected to the auxiliary insertion tool 3 between the suction unit 41 and the liquid supplying unit 42 by operation of the practitioner. The switching valve 43 may be an electromagnetic valve which electromagnetically acts under the control of the control device 6, for example.

The syringe unit 44 is connected to the auxiliary insertion tool 3 through the joint part 315, the tube 46, the switching valve 43, and the on-off valve 45. The syringe unit 44 supplies a medical agent into the subject. Here, the medical agent is steroid, an antibacterial agent, or the like. A temperature responsive gel or the like having a higher viscosity as the temperature approaches a body temperature may be mixed with the medical agent to be used. In this case, when the medical agent is applied to the affected part of the subject, the viscosity of the medical agent is increased due to the body temperature of the subject, and the medical agent is unlikely to flow from the affected part of the subject. As a result, residence time of the medical agent can be prolonged.

The on-off valve 45 includes a three-way cock or the like. The on-off valve 45 is connected to each of the liquid supplying unit 42, the switching valve 43, and the syringe unit 44. The on-off valve 45 selectively switches the part connected to the auxiliary insertion tool 3 between the syringe unit 44 and the liquid supplying unit 42 by operation of the practitioner. The on-off valve 45 may be an electromagnetic valve which electromagnetically acts under the control of the control device 6, for example.

Next, a structure of the display device 5 will be described. The display device 5 displays an image captured by the endoscope 2 and various pieces of information on the endoscope system 1 under the control of the control device 6. The display device 5 includes a display panel of, for example, a liquid crystal or organic electro luminescence (EL).

The control device 6 (processor) totally controls elements included in the endoscope system 1. In addition, the control device 6 performs image processing for the image captured by the endoscope 2 to display the image in the display device 5. The control device 6 includes a central processing unit (CPU), a nonvolatile memory, a volatile memory, or the like.

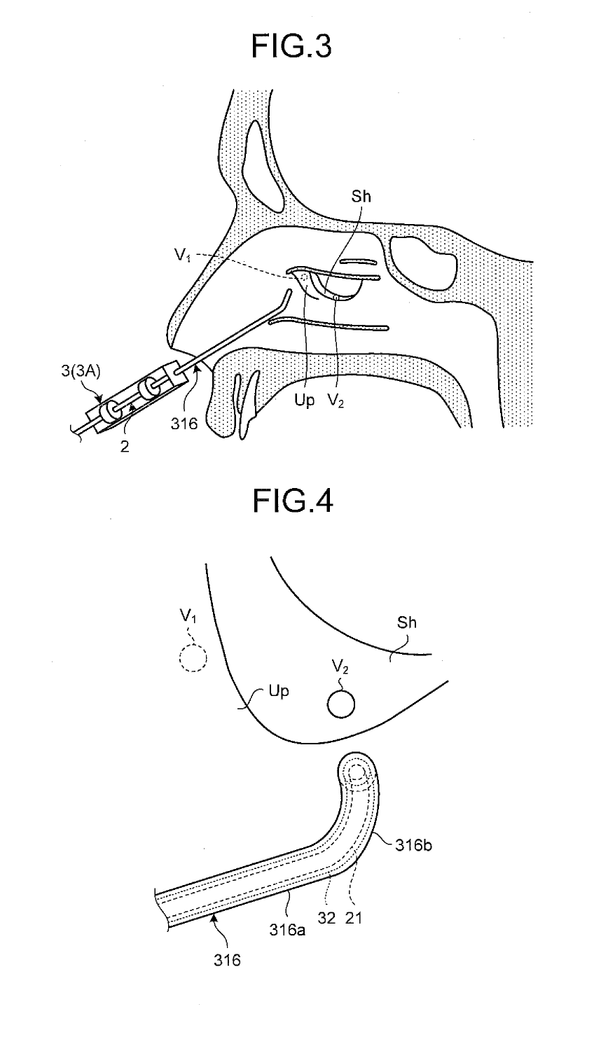

Next, reference will be made to a treatment which is performed while the inside of the maxillary sinus of the subject is observed using the above-described endoscope 2 and guide sheath 32. As illustrated in FIG. 3, there exist two openings leading to the maxillary sinus in the nasal cavity. The first embodiment is described by assuming that the endoscope 2 is inserted into one of the two openings (a first opening leading to maxillary sinus V₁ and a second opening leading to maxillary sinus V₂ illustrated in FIG. 3) leading to the maxillary sinus (maxillary sinus Ms illustrated in FIG. 42), and the guide sheath 32 is inserted into the other opening. The first opening leading to maxillary sinus V₁ is referred to as a second opening, and the second opening leading to maxillary sinus V₂ is referred to as a first opening. In general, the first opening leading to maxillary sinus V₁ may be referred to as a natural ostium, and the second opening leading to maxillary sinus V₂ may be referred to as an accessory ostium.

First, reference will be made to a method for inserting the endoscope 2 and the guide sheath 32 into the maxillary sinus Ms through the second opening leading to maxillary sinus V₂. FIGS. 3 to 8 are schematic views illustrating a treatment method for a maxillary sinus according to the first embodiment of the present invention. FIGS. 3 to 8 schematically illustrate a method for inserting the endoscope 2 and the guide sheath 32 into the second opening leading to maxillary sinus V₂ while the inside of the nasal cavity of the subject is observed using the endoscope 2 and the guide sheath 32.

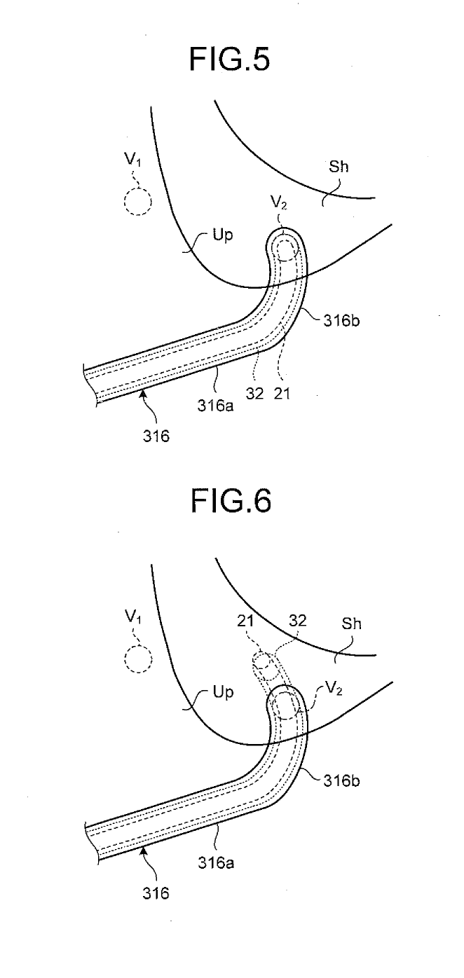

The practitioner inserts the distal end of the guide pipe 316 into the middle nasal meatus (refer to FIG. 3) by operating the handle unit 31 of the auxiliary insertion tool 3 (first auxiliary insertion tool 3A) to place the distal end of the guide pipe 316 near the second opening leading to maxillary sinus V₂ leading to the maxillary sinus Ms (refer to FIGS. 4 and 5). At this time, the inside of the nasal cavity is observed using an image captured by the endoscope 2. Here, the distal end of the guide pipe 316 has a rounded and tapered shape. Therefore, the distal end part of the guide pipe 316 can be easily inserted into a semilunar hiatus Sh or the like.

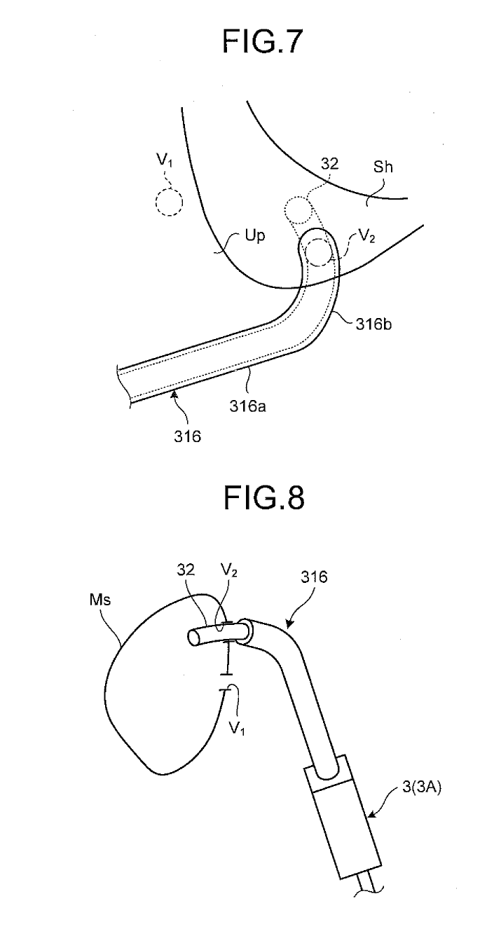

Subsequently, the practitioner operates the first operating unit 313 to insert the distal end of the guide sheath 32 into the maxillary sinus Ms through the second opening leading to maxillary sinus V₂ (refer to FIG. 6). After inserting the distal end of the guide sheath 32 into the maxillary sinus Ms, the practitioner pulls out the endoscope 2 from the guide sheath 32 (refer to FIG. 7). With the above-described treatment, only the distal end of the guide sheath 32 of the first auxiliary insertion tool 3A can be inserted into the maxillary sinus Ms through the second opening leading to maxillary sinus V₂ (refer to FIG. 8).

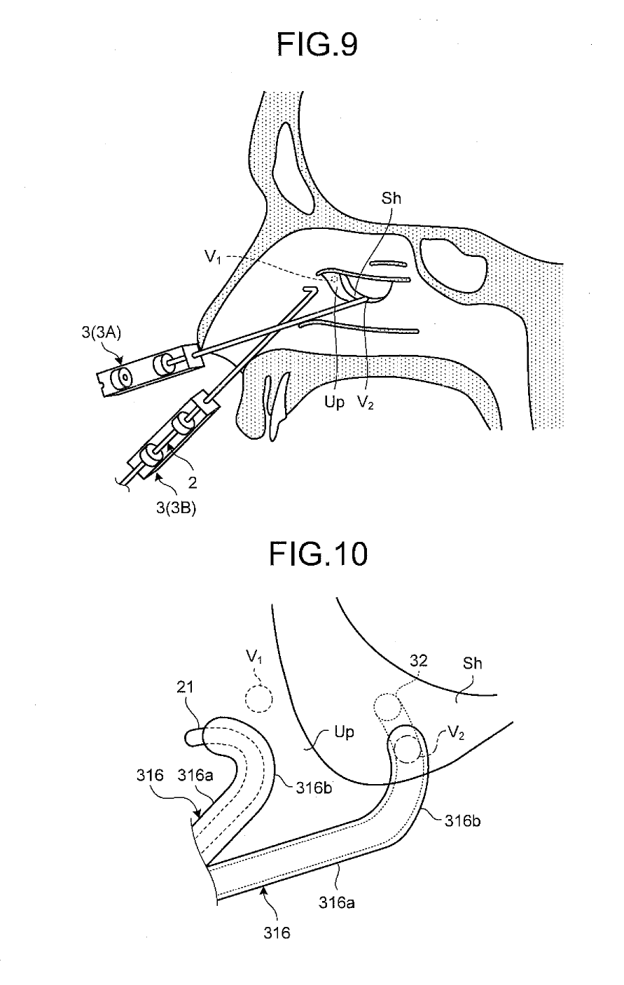

Next, reference will be made to a method for inserting the endoscope 2 (a distal end of the main body part 21 including the distal end part 20) into the maxillary sinus Ms through the first opening leading to maxillary sinus V₁. FIGS. 9 to 14 are schematic views illustrating a treatment method for a maxillary sinus according to the first embodiment of the present invention. FIGS. 9 to 14 schematically illustrate a method for inserting the endoscope 2 into the first opening leading to maxillary sinus V₁ while the inside of the nasal cavity of the subject is observed using the endoscope 2.

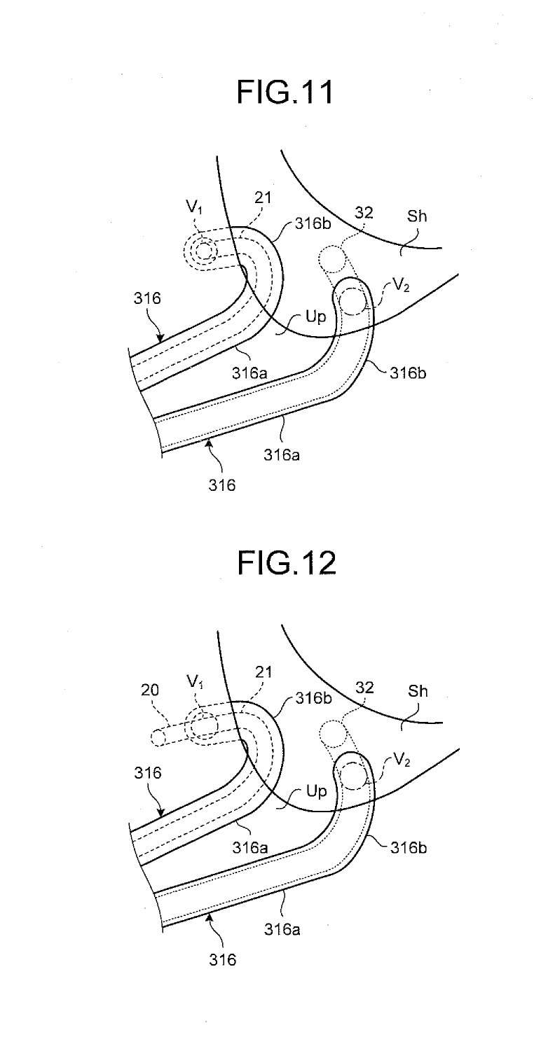

As described above, the practitioner inserts the distal end of the guide pipe 316 into the middle nasal meatus (refer to FIG. 9) by operating the handle unit 31 of the auxiliary insertion tool 3 (second auxiliary insertion tool 3B) to place the distal end of the guide pipe 316 near the first opening leading to maxillary sinus V₁ leading to the paranasal sinus (maxillary sinus Ms) through the semilunar hiatus Sh (refer to FIGS. 10 and 11). Also in this case, the inside of the nasal cavity is observed using an image obtained with the endoscope 2. At this time, as the endoscope 2 used for the observation, for example, the endoscope 2 used for inserting the guide sheath 32 of the first auxiliary insertion tool 3A into the second opening leading to maxillary sinus V₂, that is, the endoscope 2 pulled out in a state illustrated in FIG. 7 is used. In addition, the guide pipe 316 of the second auxiliary insertion tool 3B is preferably bent depending on a position of the first opening leading to maxillary sinus V₁. The bent pipe part 316b of the guide pipe 316 of the second auxiliary insertion tool 3B may be bent differently in appearance from the bent pipe part 316b of the guide pipe 316 of the first auxiliary insertion tool 3A. For example, in the first auxiliary insertion tool 3A to access the second opening leading to maxillary sinus V₂, an angle between a central axis of the straight pipe part 316a and a central axis (axis parallel to a tangent) of the distal end of the bent pipe part 316b is 70° to 90°. In the second auxiliary insertion tool 3B to access the first opening leading to maxillary sinus V₁, the angle between the central axis of the straight pipe part 316a and the central axis (axis parallel to a tangent) of the distal end of the bent pipe part 316b is approximately 110°. The guide pipe 316 of the first auxiliary insertion tool 3A may be different from the guide pipe 316 of the second auxiliary insertion tool 3B, for example, in thickness, length, material, or the like.

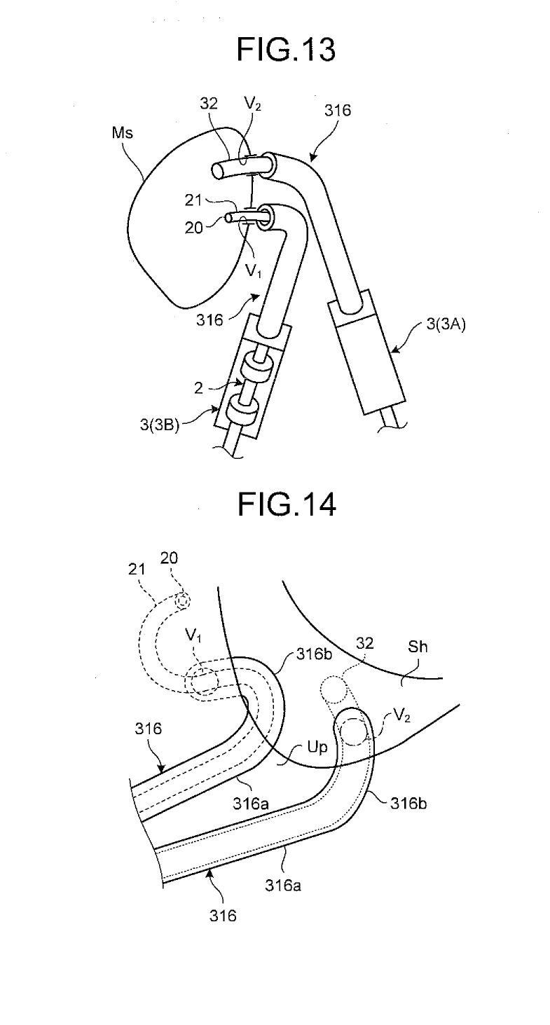

Subsequently, the practitioner operates the second operating unit 314 to make the endoscope 2 protrude from the distal end of the guide pipe 316. The practitioner then inserts the distal end of the main body part 21 including the distal end part 20 into the maxillary sinus Ms through the first opening leading to maxillary sinus V₁ (refer to FIG. 12). With the above-described treatment, the distal end of the main body part 21 including the distal end part 20 of the endoscope 2 and inserted into the guide pipe 316 of the second auxiliary insertion tool 3B can be inserted into the maxillary sinus Ms through the first opening leading to maxillary sinus V₁ (refer to FIG. 13).

At this time, the practitioner operates the second operating unit 314 or the like to adjust a direction of the distal end part 20. The practitioner places the distal end part 20 such that the distal end part 20 faces the distal end of the guide sheath 32 of the first auxiliary insertion tool 3A inserted into the maxillary sinus Ms through the second opening leading to maxillary sinus V₂ and a treatment target site.

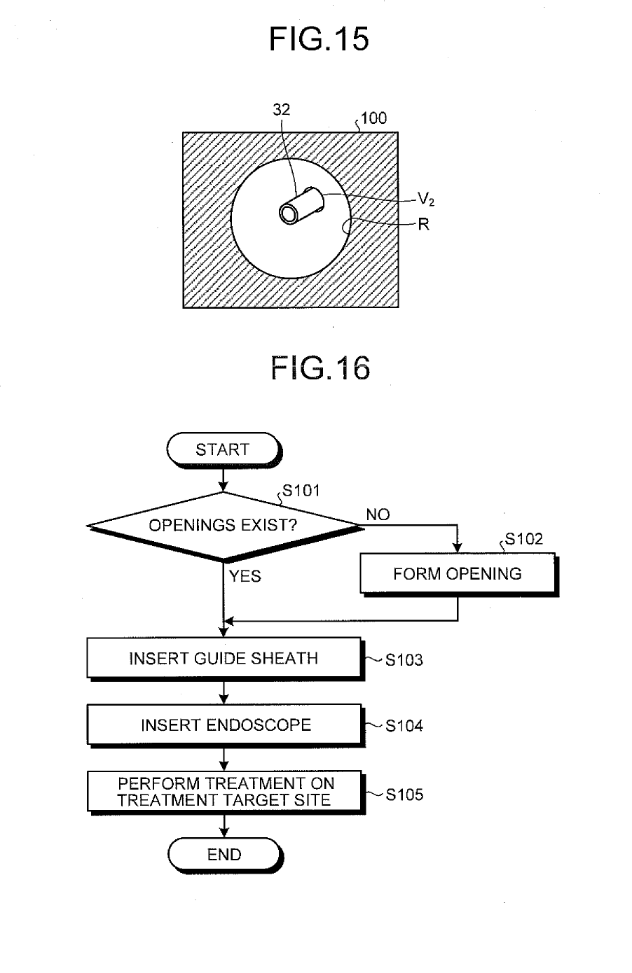

FIG. 15 is a schematic view illustrating the treatment method for a maxillary sinus according to the first embodiment of the present invention, and schematically illustrates an image captured by the endoscope 2 inserted into the second auxiliary insertion tool 3B. An image 100 illustrated in FIG. 15 is displayed on the display device 5. Specifically, an image of an area R corresponding to a visual field of the endoscope 2 is displayed on the display device 5. The image 100 including an image in the maxillary sinus Ms, including the distal end of the guide sheath 32 and the treatment target site, is displayed in the area R. In this way, by adjusting the position of the distal end part 20 of the endoscope 2, it is possible to observe the guide sheath 32 inserted into the maxillary sinus Ms through the second opening leading to maxillary sinus V₂.

The practitioner then operates the treatment unit 4 to perform a treatment in the paranasal sinus by causing liquid or gas to be delivered to the treatment target site in the maxillary sinus Ms through the inner channel of the guide sheath 32 or by sucking liquid or the like in the maxillary sinus Ms through the inner channel of the guide sheath 32. In this way, the practitioner can perform a treatment in the paranasal sinus while watching the image 100 in the paranasal sinus displayed on the display device 5.

In the above-described treatment method for a maxillary sinus, when the first opening leading to maxillary sinus V₁ or the second opening leading to maxillary sinus V₂ does not exist, the first opening leading to maxillary sinus V₁ or the second opening leading to maxillary sinus V₂ may be formed using a treatment tool such as a knife, a curette, a puncture needle, or a probe. Before the above-described treatment method is performed, it is checked whether the first opening leading to maxillary sinus V₁ and the second opening leading to maxillary sinus V₂ exist using the endoscope 2 or a rigid endoscope 300 described later. For example, if the second opening leading to maxillary sinus V₂ does not exist, treatment for forming the second opening leading to maxillary sinus V₂ is performed.

Here, procedures of the above-described treatment method for a maxillary sinus will be described with reference to FIG. 16. FIG. 16 is a flowchart illustrating the treatment method for a maxillary sinus according to the first embodiment of the present invention. First, the practitioner checks whether the first opening leading to maxillary sinus V₁ and the second opening leading to maxillary sinus V₂ exist using the endoscope 2 or another endoscope (for example, the rigid endoscope 300) (Step S101). When confirming that the first opening leading to maxillary sinus V₁ and the second opening leading to maxillary sinus V₂ exist (Step S101: Yes), the practitioner performs a treatment of Step S103.

On the other hand, when confirming that one of the first opening leading to maxillary sinus V₁ and the second opening leading to maxillary sinus V₂ does not exist (Step S101: No), the practitioner performs a treatment of Step S102. In Step S102, the practitioner performs a treatment for forming one of the first opening leading to maxillary sinus V₁ and the second opening leading to maxillary sinus V₂, which does not exist (Step S102). After the treatment for forming the opening, the practitioner performs a treatment of Step S103.

In Step S103, the practitioner inserts the guide sheath 32 (first sheath) into the maxillary sinus Ms through the second opening leading to maxillary sinus V₂ (first opening) leading to the maxillary sinus Ms in the nasal cavity using the first auxiliary insertion tool 3A (Step S103). The practitioner then pulls out the endoscope 2 from the first auxiliary insertion tool 3A (guide sheath 32) to insert the endoscope 2 into the second auxiliary insertion tool 3B.

Subsequently, the practitioner inserts the endoscope 2 into the maxillary sinus Ms through the first opening leading to maxillary sinus V₁ (second opening) leading to the maxillary sinus Ms using the second auxiliary insertion tool 3B (Step S104). In Steps S101 and S102, the inserting operation is performed while imaging is performed with the endoscope 2 and the image obtained by the imaging is checked in the display device 5.

The practitioner then performs a treatment on the treatment target site in the maxillary sinus Ms while the guide sheath 32 and the treatment target site exist in the visual field of the endoscope 2 (Step S105). In the first embodiment, for example, the treatment target site is the inside of the maxillary sinus Ms. Pus or the like accumulated in the maxillary sinus Ms is sucked using the guide sheath 32, or a medical agent is delivered thereto after sucking.

According to the above-described first embodiment, the endoscope 2 and the guide sheath 32 are inserted into the maxillary sinus Ms through the two different openings (first opening leading to maxillary sinus V₁ and second opening leading to maxillary sinus V₂) leading to the maxillary sinus Ms while imaging is performed with the endoscope 2. Therefore, minimally invasive observation or treatment of the paranasal sinus can be performed. In the above-described related art, an opening is enlarged by an expandable body such as a balloon, in which case a bone or the like around the opening may be broken when the opening is enlarged. On the other hand, according to the first embodiment, the endoscope 2 and the guide sheath 32 can be inserted into the maxillary sinus Ms without enlarging the opening to perform a minimally invasive treatment.

The above-described first embodiment has been described by assuming that the endoscope 2 is inserted into the first opening leading to maxillary sinus V₁, and the guide sheath 32 is inserted into the second opening leading to maxillary sinus V₂ when the treatment for the treatment target site is performed. However, the guide sheath 32 may be inserted into the first opening leading to maxillary sinus V₁, and the endoscope 2 may be inserted into the second opening leading to maxillary sinus V₂. In this case, the first opening leading to maxillary sinus V₁ is referred to as a first opening, and the second opening leading to maxillary sinus V₂ is referred to as a second opening.

### First Modified Example of First Embodiment

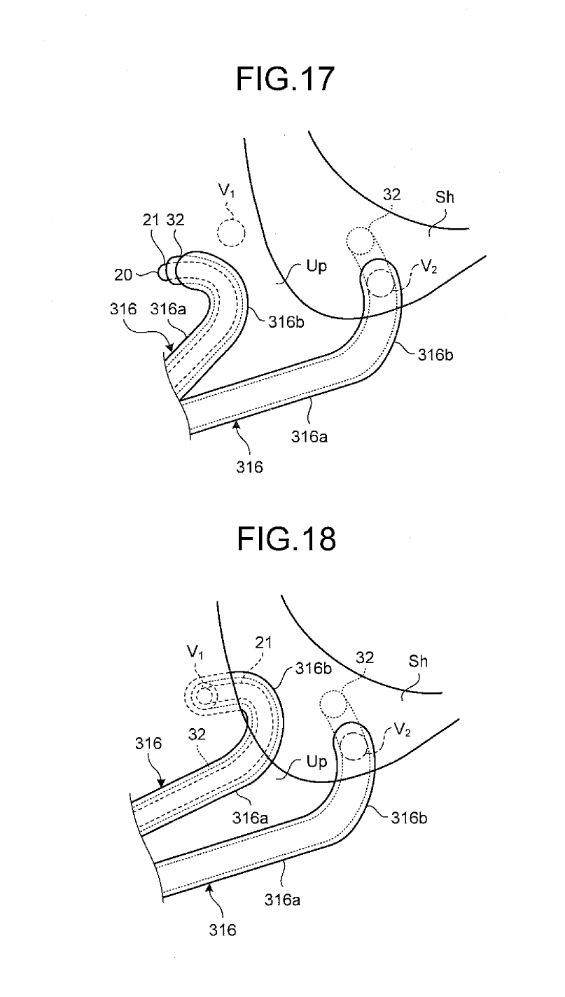

Next, a first modified example of the first embodiment will be described with reference to FIGS. 17 to 20. FIGS. 17 to 20 are schematic views illustrating a treatment method for a maxillary sinus according to the first modified example of the first embodiment of the present invention, and schematically illustrate a method for inserting the endoscope 2 and the guide sheath 32 into the first opening leading to maxillary sinus V₁ while the inside of the nasal cavity of the subject is observed using the endoscope 2 and the guide sheath 32. The above-described first embodiment has been described by assuming that only the main body part 21 of the endoscope 2 is inserted into the maxillary sinus Ms through the first opening leading to maxillary sinus V₁. However, in the first modified example, the guide sheath 32 into which the endoscope 2 has been inserted is inserted into the maxillary sinus Ms through the first opening leading to maxillary sinus V₁. The first modified example is described by assuming that the guide sheath 32 which has been inserted into the maxillary sinus Ms through the second opening leading to maxillary sinus V₂ is referred to as a first sheath, and the guide sheath 32 which has been inserted into the maxillary sinus Ms through the first opening leading to maxillary sinus V₁ is referred to as a second sheath.

As in the above-described first embodiment, the practitioner places the distal end of the guide pipe 316 near the first opening leading to maxillary sinus V₁ leading to the maxillary sinus Ms by operating the handle unit 31 of the auxiliary insertion tool 3 (second auxiliary insertion tool 3B) while the distal end of the guide sheath (first sheath) of the first auxiliary insertion tool 3A is inserted into the maxillary sinus Ms through the second opening leading to maxillary sinus V₂ (refer to FIGS. 17 and 18). At this time, as the endoscope 2 (the endoscope 2 to be inserted into the guide sheath 32) used for the observation, for example, the endoscope 2 used for inserting the guide sheath 32 of the first auxiliary insertion tool 3A into the second opening leading to maxillary sinus V₂, that is, the endoscope 2 pulled out in the state illustrated in FIG. 7 is used.

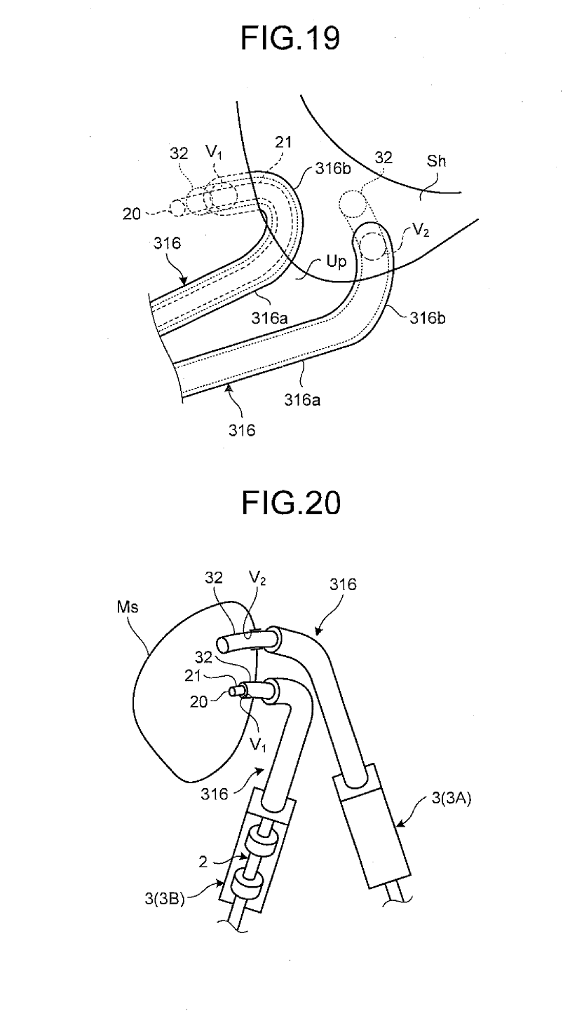

Subsequently, the practitioner operates the first operating unit 313 of the second auxiliary insertion tool 3B to insert the distal end of the guide sheath 32 (second sheath) into the maxillary sinus Ms through the first opening leading to maxillary sinus V₁ (refer to FIG. 19). With the above-described treatment, the distal end of the guide sheath 32 of the second auxiliary insertion tool 3B and the distal end of the main body part 21 of the endoscope 2 can be inserted into the maxillary sinus Ms through the first opening leading to maxillary sinus V₁ (refer to FIG. 20).

According to the above-described first modified example, the guide sheaths 32 (first sheath and second sheath) of the first and second auxiliary insertion tools 3 (3A and 3B) are inserted into the maxillary sinus Ms through the two different openings (first opening leading to maxillary sinus V₁ and second opening leading to maxillary sinus V₂) leading to the maxillary sinus Ms, respectively. Therefore, minimally invasive observation or treatment of the paranasal sinus can be performed.

In addition, according to the first modified example, the guide sheaths 32 are inserted into the maxillary sinus Ms through the two openings (first opening leading to maxillary sinus V₁ and second opening leading to maxillary sinus V₂), respectively. Therefore, the endoscope 2 can be easily inserted into either one of the guide sheaths 32. As a result, the degree of freedom between the observation with the endoscope 2 and the treatment with the guide sheath 32 can be higher than that in the above-described first embodiment.

### Second Modified Example of First Embodiment

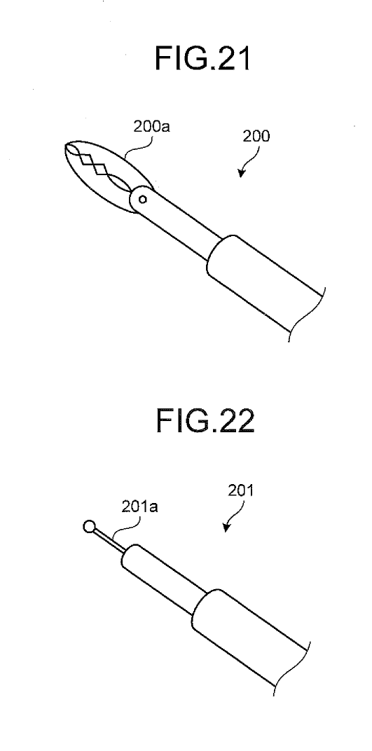

Next, a second modified example of the first embodiment will be described with reference to FIG. 21. FIG. 21 is a schematic view illustrating a treatment method for a maxillary sinus according to the second modified example of the first embodiment of the present invention, and illustrating an example of the treatment tool to be inserted into the guide sheath 32. The above-described first embodiment has been described by assuming that liquid or gas is delivered, or liquid or the like is sucked by the guide sheath 32 inserted into the maxillary sinus Ms through the second opening leading to maxillary sinus V₂. However, in the second modified example, the treatment in the paranasal sinus (in the maxillary sinus) is performed while the treatment tool protrudes from the distal end of the guide sheath 32. The second modified example and the following third to fifth modified examples are described by assuming that the treatment member includes the guide sheath 32 and the treatment tool.

A treatment tool 200 according to the second modified example is flexible and elongate, and includes a biopsy forceps 200a at a distal end thereof. By inserting the treatment tool 200 into the guide sheath 32 and making the biopsy forceps 200a protrude from the distal end of the guide sheath 32, for example, a part of a tissue in the maxillary sinus Ms can be collected. For example, opening and closing actions of the biopsy forceps 200a are operated by an operating unit (not illustrated) arranged at the proximal end of the treatment tool 200 and a wire (not illustrated) which connects the operating unit and the bioptome.

### Third Modified Example of First Embodiment

Next, a third modified example of the first embodiment will be described with reference to FIG. 22. FIG. 22 is a schematic view illustrating a treatment method for a maxillary sinus according to the third modified example of the first embodiment of the present invention, and illustrating an example of the treatment tool to be inserted into the guide sheath 32. The above-described first embodiment has been described by assuming that liquid or gas is delivered, or liquid or the like is sucked by the guide sheath 32 inserted into the maxillary sinus Ms through the second opening leading to maxillary sinus V₂. However, in the third modified example, the treatment in the paranasal sinus (in the maxillary sinus) is performed while the treatment tool protrudes from the distal end of the guide sheath 32.

A treatment tool 201 according to the third modified example is flexible and elongate, and includes a high-frequency knife 201a at a distal end thereof. By inserting the treatment tool 201 into the guide sheath 32 and making the high-frequency knife 201a protrude from the distal end of the guide sheath 32, for example, a part of a wall surface in the maxillary sinus Ms can be incised or exfoliated. For example, by operating the operating unit (not illustrated) connected to the proximal end of the treatment tool 201, the high-frequency knife 201a is switched on and off in electric conduction. In addition to the high-frequency knife, for example, an energy treatment tool emitting ultrasonic waves, laser, or the like to cauterize or sterilize the treatment target site may be used.

### Fourth Modified Example of First Embodiment

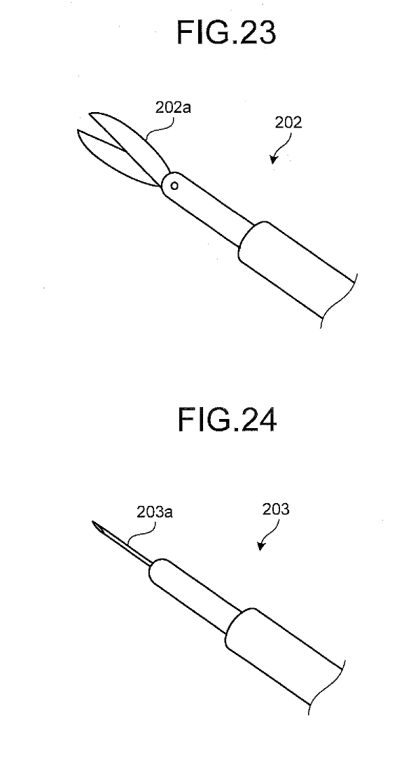

Next, a fourth modified example of the first embodiment will be described with reference to FIG. 23. FIG. 23 is a schematic view illustrating a treatment method for a maxillary sinus according to the fourth modified example of the first embodiment of the present invention, and illustrating an example of the treatment tool to be inserted into the guide sheath 32. The above-described first embodiment has been described by assuming that liquid or gas is delivered, or liquid or the like is sucked by the guide sheath 32 inserted into the maxillary sinus Ms through the second opening leading to maxillary sinus V₂. However, in the fourth modified example, the treatment in the paranasal sinus (in the maxillary sinus) is performed while the treatment tool protrudes from the distal end of the guide sheath 32.

A treatment tool 202 according to the fourth modified example is flexible and elongate, and includes a pair of scissors 202a at a distal end thereof. By inserting the treatment tool 202 into the guide sheath 32 and making the pair of scissors 202a protrude from the distal end of the guide sheath 32, for example, a part of a tissue in the maxillary sinus Ms can be excised. For example, opening and closing actions of the pair of scissors 202a are operated by an operating unit (not illustrated) arranged at the proximal end of the treatment tool 202 and a wire (not illustrated) which connects the operating unit and the bioptome.

### Fifth Modified Example of First Embodiment

Next, a fifth modified example of the first embodiment will be described with reference to FIG. 24. FIG. 24 is a schematic view illustrating a treatment method for a maxillary sinus according to the fifth modified example of the first embodiment of the present invention, and illustrating an example of the treatment tool to be inserted into the guide sheath 32. The above-described first embodiment has been described by assuming that liquid or gas is delivered, or liquid or the like is sucked by the guide sheath 32 inserted into the maxillary sinus Ms through the second opening leading to maxillary sinus V₂. However, in the fifth modified example, the treatment in the paranasal sinus (in the maxillary sinus) is performed while the treatment tool protrudes from the distal end of the guide sheath 32.

A treatment tool 203 according to the fifth modified example is flexible and elongate, and includes an injection needle 203a at a distal end thereof. By inserting the treatment tool 203 into the guide sheath 32 and making the injection needle 203a protrude from the distal end of the guide sheath 32, for example, a tissue in the maxillary sinus Ms can be punctured with the injection needle 203a, and a medical agent can be injected thereinto. For example, the injection needle 203a includes an operating unit (a syringe and a piston) (not illustrated) arranged at the proximal end of the treatment tool 203, and a tube (not illustrated) which connects the operating unit and the bioptome. The medical agent is delivered through the tube by the operation of the operating unit.

The above-described second to fifth modified examples have been described by assuming that the treatment member includes the guide sheath 32 and the treatment tool. However, the treatment tools 200 to 203 may be inserted directly into the maxillary sinus Ms, not through the guide sheath 32. In this case, the treatment member includes only the treatment tools 200 to 203.

### Second Embodiment

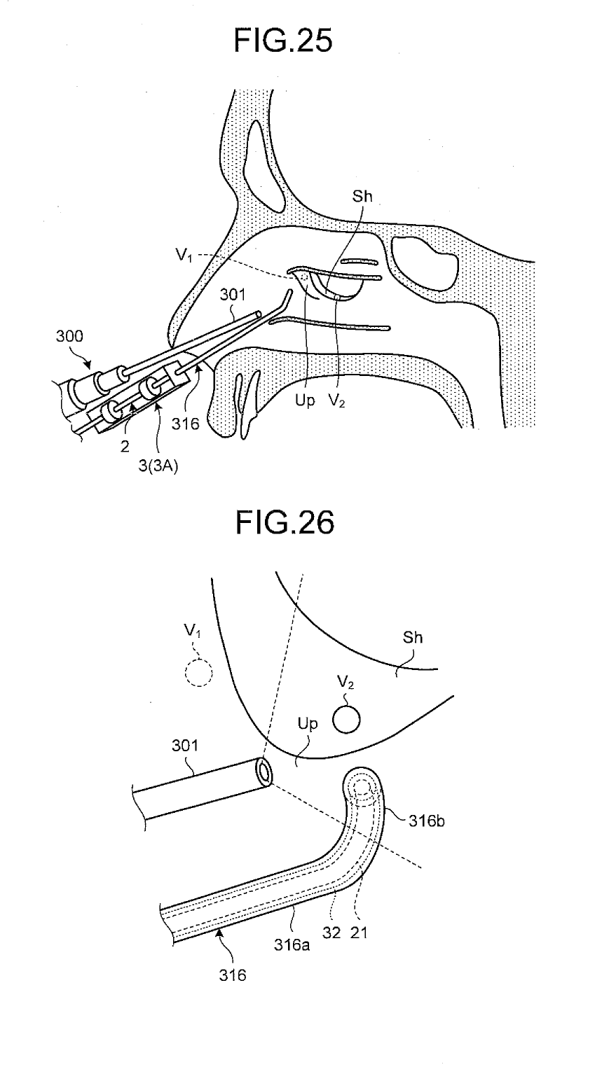

Next, a second embodiment of the present invention will be described with reference to FIGS. 25 to 33. The same reference signs are given to the same elements as the above-described elements. The above-described first embodiment has been described by assuming that the guide sheath 32 and the endoscope 2 are inserted into the maxillary sinus Ms while the inside of the nasal cavity is observed using the endoscope 2 inserted into the guide sheath 32. However, in the second embodiment, the guide sheath 32 and the endoscope 2 are inserted into the maxillary sinus Ms while the inside of the nasal cavity is observed using a second endoscope (hereinafter, referred to as a rigid endoscope) different from the endoscope 2 (for example, referred to as a first endoscope).

First, reference will be made to a method for inserting the endoscope 2 and the guide sheath 32 into the maxillary sinus Ms through the second opening leading to maxillary sinus V₂. FIGS. 25 to 29 are schematic views illustrating a treatment method for a maxillary sinus according to the second embodiment of the present invention, and schematically illustrate a method for inserting the endoscope 2 and the guide sheath 32 into the second opening leading to maxillary sinus V₂ while the inside of the nasal cavity of the subject is observed using a rigid endoscope 300. For example, a rigid endoscope is used as the rigid endoscope 300. The rigid endoscope 300 captures an image in the nasal cavity through an optical system arranged at the distal end of an insertion part 301. An image obtained by the imaging is displayed on a display device. That is, the display device displays the image obtained by the rigid endoscope 300 and the image obtained by the endoscope 2 simultaneously. In addition to the rigid endoscope 300, a flexible endoscope or an endoscope 2 of the same kind, different from the endoscope 2 to be inserted into the maxillary sinus Ms may be used as the second endoscope.

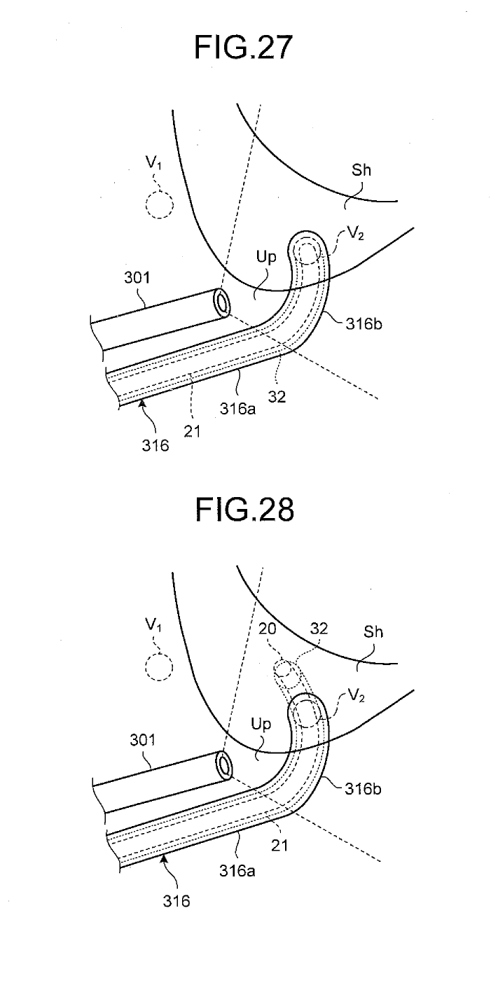

The practitioner inserts the distal end of the guide pipe 316 into the middle nasal meatus (refer to FIG. 25) to place the distal end of the guide pipe 316 near the second opening leading to maxillary sinus V₂ leading to the paranasal sinus (maxillary sinus Ms) by operating the handle unit 31 of the first auxiliary insertion tool 3A under observation with the rigid endoscope 300 and the endoscope 2 while the distal end of the guide pipe 316 of the auxiliary insertion tool 3 (first auxiliary insertion tool 3A) is included in a visual field of the rigid endoscope 300 (refer to FIGS. 26 and 27). At this time, the distal end of the guide pipe 316 may access the vicinity of the second opening leading to maxillary sinus V₂ after it is confirmed that the distal end of the guide pipe 316 is included in the visual field (image) by inserting the rigid endoscope 300 into the nasal cavity, and then inserting the guide pipe 316 thereinto. Alternatively, the distal end of the guide pipe 316 may access the vicinity of the second opening leading to maxillary sinus V₂ while the guide pipe 316 is placed in the visual field by inserting the guide pipe 316 into the nasal cavity to some extent, and then inserting the rigid endoscope 300 thereinto.

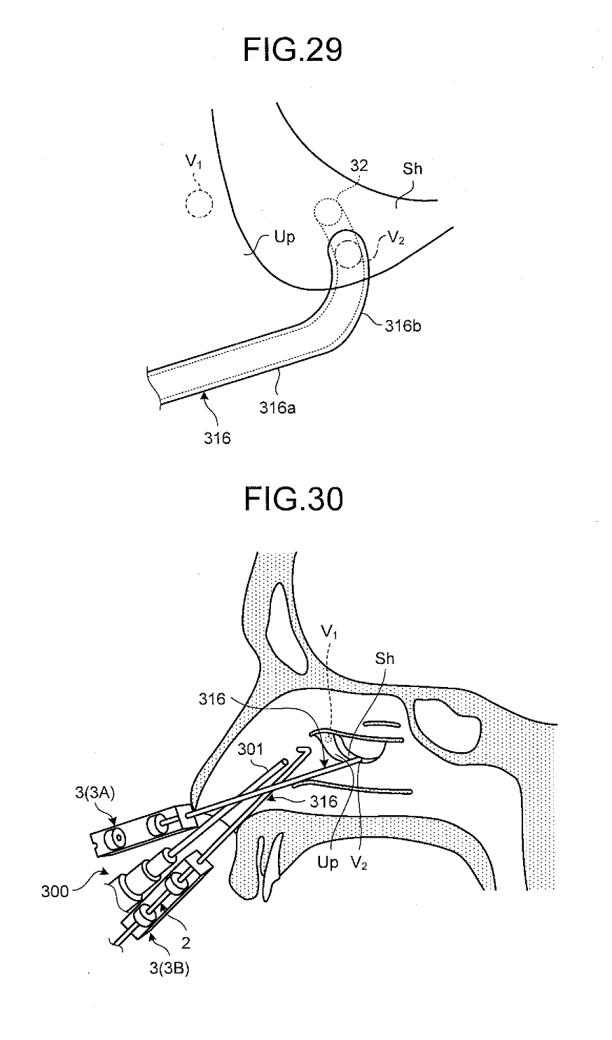

Subsequently, the practitioner operates the first operating unit 313 to insert the distal end of the guide sheath 32 into the maxillary sinus Ms through the second opening leading to maxillary sinus V₂ (refer to FIG. 28). After inserting the distal end of the guide sheath 32 into the maxillary sinus Ms, the practitioner pulls out the endoscope 2 from the guide sheath 32 (refer to FIG. 29). With the above-described treatment, only the distal end of the guide sheath 32 of the first auxiliary insertion tool 3A can be inserted into the maxillary sinus Ms through the second opening leading to maxillary sinus V₂ (for example, refer to FIG. 8). As in the above-described second embodiment, the guide sheath 32 can be easily inserted into the maxillary sinus Ms through the second opening leading to maxillary sinus V₂ by displaying a wide range of visual field (image) with the rigid endoscope 300 and displaying a local visual field (image) with the endoscope 2.

Next, reference will be made to a method for inserting the endoscope 2 (a distal end of the main body part 21 including the distal end part 20) into the maxillary sinus Ms through the first opening leading to maxillary sinus V₁. FIGS. 30 to 33 are schematic views illustrating a treatment method for a maxillary sinus according to the second embodiment of the present invention, and schematically illustrate a method for inserting the endoscope 2 into the first opening leading to maxillary sinus V₁ while the inside of the nasal cavity of the subject is observed using the rigid endoscope 300.

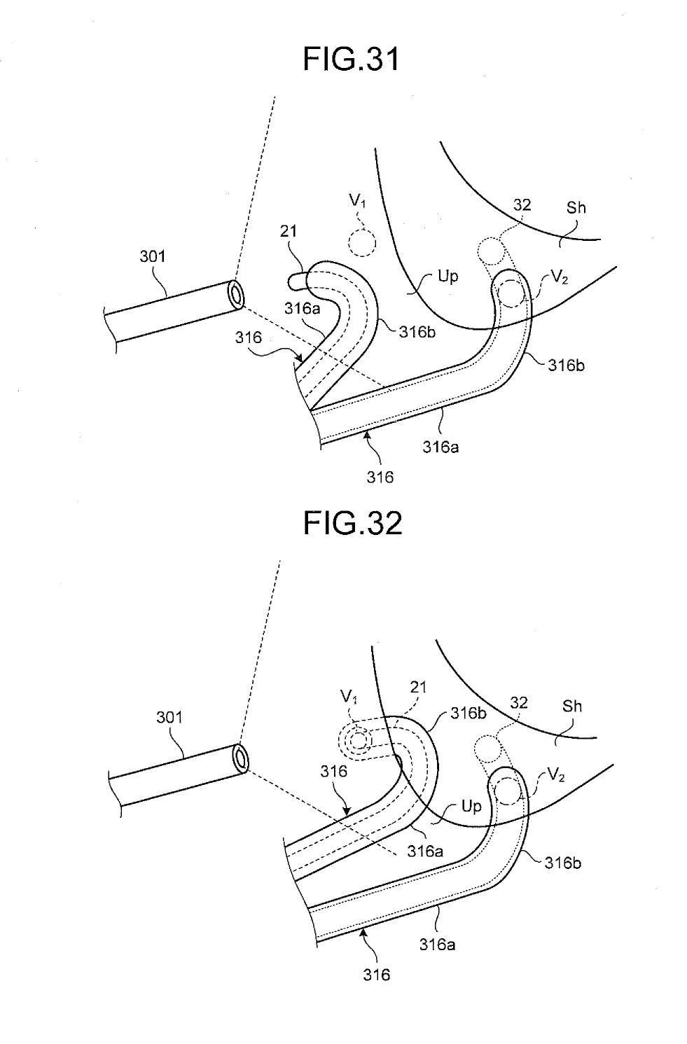

As described above, the practitioner inserts the distal end of the guide pipe 316 into the middle nasal meatus (refer to FIG. 30) to place the distal end of the guide pipe 316 near the first opening leading to maxillary sinus V₁ leading to the paranasal sinus (maxillary sinus Ms) by operating the handle unit 31 of the second auxiliary insertion tool 32 under observation with the rigid endoscope 300 and the endoscope 2 while the distal end of the guide pipe 316 of the auxiliary insertion tool 3 (second auxiliary insertion tool 3B) is included in the visual field of the rigid endoscope 300 (refer to FIGS. 31 and 32).

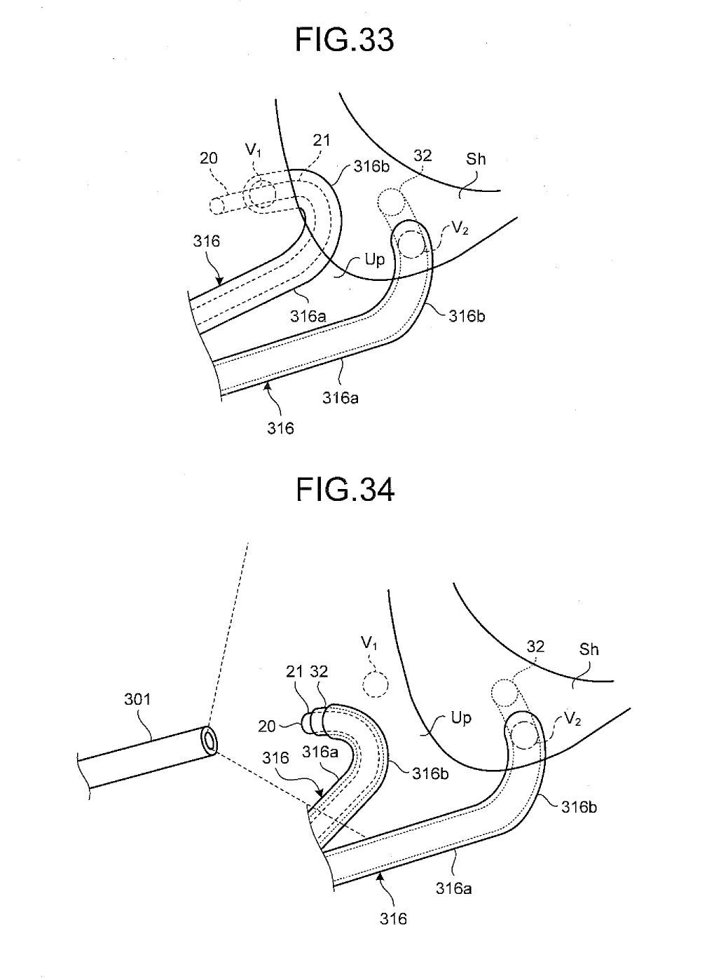

Subsequently, the practitioner operates the second operating unit 314 to make the endoscope 2 protrude from the distal end of the guide pipe 316. The practitioner then inserts the distal end of the main body part 21 including the distal end part 20 into the maxillary sinus Ms through the first opening leading to maxillary sinus V₁ (refer to FIG. 33). At this time, the rigid endoscope 300 may be kept inserted into the nasal cavity, or may be pulled out from the nasal cavity. With the above-described treatment, the distal end of the main body part 21 including the distal end part 20 of the endoscope 2 and inserted into the guide pipe 316 of the second auxiliary insertion tool 3B can be inserted into the maxillary sinus Ms through the first opening leading to maxillary sinus V₁ (for example, refer to FIG. 13).

As described above, the practitioner then operates the second operating unit 314 or the like to observe the guide sheath 32 inserted into the maxillary sinus Ms by adjusting the position of the distal end part 20 such that the distal end of the guide sheath 32 is included in the visual field of the endoscope 2. The practitioner operates the treatment unit 4 to perform a treatment in the paranasal sinus by causing liquid or gas to be delivered into the maxillary sinus Ms through the inner channel of the guide sheath 32 or by sucking liquid or the like in the maxillary sinus Ms through the inner channel of the guide sheath 32.

According to the above-described second embodiment, the endoscope 2 and the guide sheaths 32 are easily inserted into the maxillary sinus Ms through the two different openings (first opening leading to maxillary sinus V₁ and second opening leading to maxillary sinus V₂) leading to the maxillary sinus Ms under observation with the rigid endoscope 300.

The above-described second embodiment has been described by assuming that the guide sheath 32 is inserted into the second opening leading to maxillary sinus V₂, and then the endoscope 2 is inserted into the first opening leading to maxillary sinus V₁. However, the endoscope 2 may be inserted into the first opening leading to maxillary sinus V₁, and then the guide sheath 32 may be inserted into the second opening leading to maxillary sinus V₂. As in the above-described first embodiment, the guide sheath 32 may be inserted into the first opening leading to maxillary sinus V₁, and the endoscope 2 may be inserted into the second opening leading to maxillary sinus V₂. In this case, the first opening leading to maxillary sinus V₁ is referred to as a first opening, and the second opening leading to maxillary sinus V₂ is referred to as a second opening.

### First Modified Example of Second Embodiment

Next, a first modified example of the second embodiment will be described with reference to FIGS. 34 to 37. FIGS. 34 to 37 are schematic views illustrating a treatment method for a maxillary sinus according to the first modified example of the second embodiment of the present invention, and schematically illustrate a method for inserting the endoscope 2 and the guide sheath 32 into the first opening leading to maxillary sinus V₁ while the inside of the nasal cavity of the subject is observed using the rigid endoscope 300. The above-described second embodiment has been described by assuming that only the main body part 21 of the endoscope 2 is inserted into the maxillary sinus Ms through the first opening leading to maxillary sinus V₁. However, in the first modified example, the guide sheath 32 into which the endoscope 2 has been inserted is inserted into the maxillary sinus Ms through the first opening leading to maxillary sinus V₁.

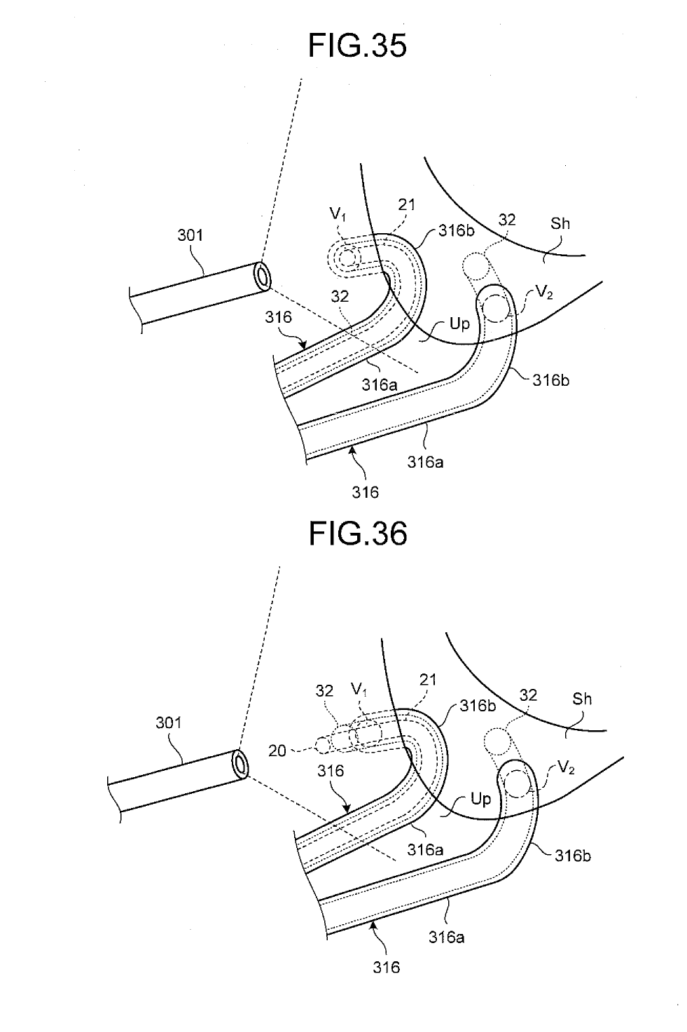

As in the above-described second embodiment, the practitioner places the distal end of the guide pipe 316 near the first opening leading to maxillary sinus V₁ leading to the paranasal sinus (maxillary sinus Ms) by operating the handle unit 31 of the auxiliary insertion tool 3 (second auxiliary insertion tool 3B) under observation with the rigid endoscope 300 while only the distal end of the guide sheath 32 of the first auxiliary insertion tool 3A is inserted into the maxillary sinus Ms through the second opening leading to maxillary sinus V₂ (refer to FIGS. 34 and 35).

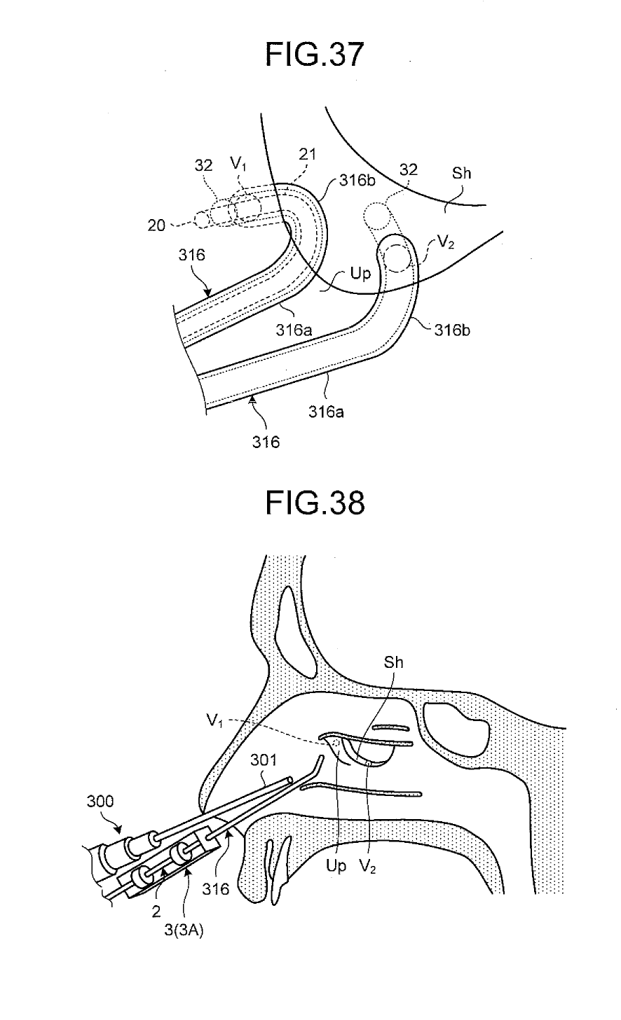

Subsequently, the practitioner operates the first operating unit 313 to insert the distal end of the guide sheath 32 into the maxillary sinus Ms through the first opening leading to maxillary sinus V₁ (refer to FIG. 36). Thereafter, by pulling out the rigid endoscope 300 (insertion part 301) from the nasal cavity, the distal end of the guide sheath 32 of the second auxiliary insertion tool 3B and the distal end of the main body part 21 of the endoscope 2 can be inserted into the maxillary sinus Ms through the first opening leading to maxillary sinus V₁ (refer to FIG. 37).

According to the above-described first modified example, the guide sheaths 32 of the first and second auxiliary insertion tools 3 (3A and 3B) are inserted into the maxillary sinus Ms through the two different openings (first opening leading to maxillary sinus V₁ and second opening leading to maxillary sinus V₂) leading to the maxillary sinus Ms under observation with the rigid endoscope 300, respectively. Therefore, minimally invasive observation or treatment of the paranasal sinus can be performed.

In addition, according to the first modified example, the guide sheaths 32 are inserted into the maxillary sinus Ms through the two openings (first opening leading to maxillary sinus V₁ and second opening leading to maxillary sinus V₂), respectively. Therefore, the endoscope 2 can be easily inserted into either one of the guide sheaths 32. As a result, the degree of freedom between the observation with the endoscope 2 and the treatment with the guide sheath 32 can be higher than that in the above-described second embodiment.

### Second Modified Example of Second Embodiment

Next, a second modified example of the second embodiment will be described with reference to FIGS. 38 to 41. FIGS. 38 to 41 are schematic views illustrating a treatment method for a maxillary sinus according to the second modified example of the second embodiment of the present invention, and schematically illustrate a method for inserting the guide sheath 32 into the second opening leading to maxillary sinus V₂ while the inside of the nasal cavity of the subject is observed using the rigid endoscope 300. The above-described second embodiment has been described by assuming that the guide sheath 32 into which the endoscope 2 has been inserted is inserted into the maxillary sinus Ms through the second opening leading to maxillary sinus V₂. However, in the second modified example, only the guide sheath 32 is inserted into the maxillary sinus Ms through the second opening leading to maxillary sinus V₂. That is, in the second modified example, the endoscope 2 is not inserted into the guide sheath 32.

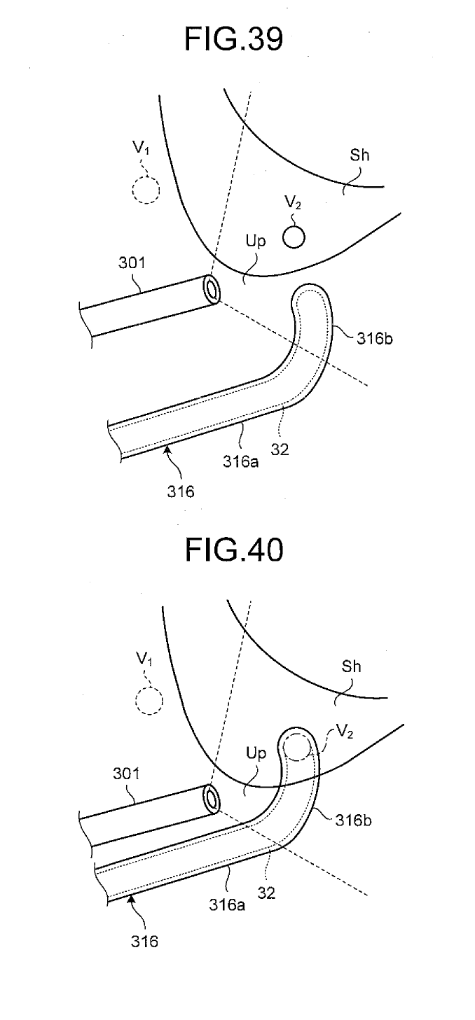

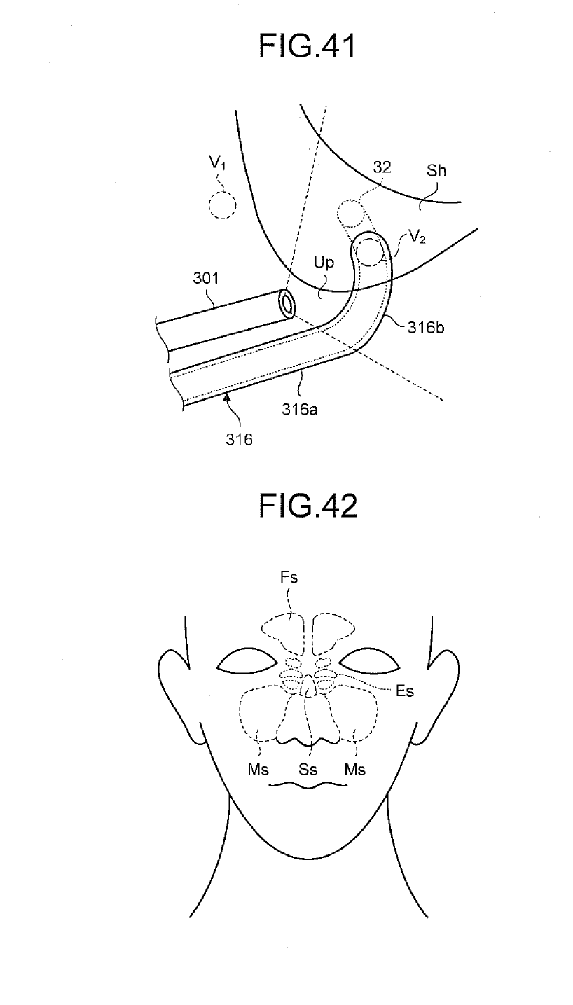

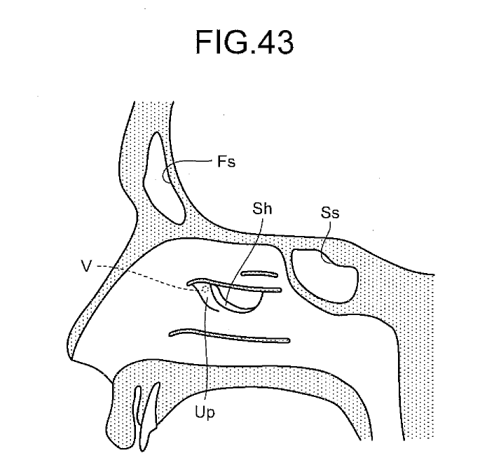

As in the above-described second embodiment, the practitioner inserts the distal end of the guide pipe 316 into the middle nasal meatus (refer to FIG. 38) to place the distal end of the guide pipe 316 near the second opening leading to maxillary sinus V₂ leading to the paranasal sinus (maxillary sinus Ms) by operating the handle unit 31 of the first auxiliary insertion tool 3A under observation with the rigid endoscope 300 while the distal end of the guide pipe 316 of the auxiliary insertion tool 3 (first auxiliary insertion tool 3A) is included in the visual field of the rigid endoscope 300 (refer to FIGS. 39 and 40). Subsequently, the practitioner operates the first operating unit 313 to insert the distal end of the guide sheath 32 into the maxillary sinus Ms through the second opening leading to maxillary sinus V₂ (refer to FIG. 41). With the above-described treatment, only the distal end of the guide sheath 32 of the first auxiliary insertion tool 3A can be inserted into the maxillary sinus Ms through the second opening leading to maxillary sinus V₂ (for example, refer to FIG. 8).

According to the above-described second modified example, the guide sheath 32 of the first auxiliary insertion tool 3A, into which the endoscope 2 has not been inserted, is inserted into the maxillary sinus Ms through one (the second opening leading to maxillary sinus V₂ in the second modified example) of the two different openings (first opening leading to maxillary sinus V₁ and second opening leading to maxillary sinus V₂) leading to the maxillary sinus Ms under observation with the rigid endoscope 300. Therefore, minimally invasive observation or treatment of the paranasal sinus can be performed.

In the above-described second embodiment and first and second modified examples, the treatment tools 200 to 203 illustrated in the second to fifth modified examples of the first embodiment may be inserted into the guide sheath 32 to perform a treatment in the paranasal sinus.

In addition, the above-described first and second embodiments and modified examples have been described by assuming that the endoscope 2 and the guide sheath 32 are brought close to the first opening leading to maxillary sinus V₁ and the second opening leading to maxillary sinus V₂ using the auxiliary insertion tool 3. However, an auxiliary insertion tool different from the auxiliary insertion tool 3, for example, an auxiliary insertion tool including a guide member to guide a traveling direction of the guide sheath 32 and a moving unit to hold the guide sheath 32 and be able to move along the guide member may be used. Only the guide sheath 32 or only the endoscope 2 may be inserted without the auxiliary insertion tool.

Furthermore, in the above-described first and second embodiments and modified examples, when there are three or more openings leading to the maxillary sinus Ms, two openings are selected appropriately, and the endoscope 2 and the guide sheath 32 are inserted into the maxillary sinus Ms through the selected openings, respectively.

The present invention is not limited to the above-described first and second embodiments and modified examples, and may include various embodiments in a range not departing from the technical idea described in the claims. In addition, the first and second embodiments and modified examples may be combined together appropriately.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A treatment method comprising:
inserting a treatment member into a maxillary sinus through a first opening in a nasal cavity leading to the maxillary sinus;
inserting an endoscope into the maxillary sinus through a second opening leading to the maxillary sinus and different from the first opening; and
performing a treatment on a treatment target site in the maxillary sinus while the treatment member and the treatment target site exist within a visual field of the endoscope.

2. The treatment method according to claim 1, wherein
the treatment member is a sheath configured to be inserted into the maxillary sinus through the first opening, and
the treatment is performed on the treatment target site after the sheath is inserted into the maxillary sinus.

3. The treatment method according to claim 1, wherein
the inserting of the endoscope into the maxillary sinus includes:
inserting a sheath into the maxillary sinus through the second opening;
inserting the endoscope into the sheath; and
making the endoscope protrude from a distal end of the sheath.

4. The treatment method according to claim 1, wherein
the treatment member and the endoscope are inserted into the maxillary sinus through the first and second openings, respectively, while imaging is performed by the endoscope.

5. The treatment method according to claim 1, wherein
the treatment member and the endoscope are inserted into the maxillary sinus through the first and second openings, respectively, under observation with a second endoscope different from the endoscope.

6. The treatment method according to claim 1, wherein
the treatment member includes a sheath configured to be inserted into the maxillary sinus through the first opening and a treatment tool configured to be inserted into the sheath,
the treatment tool is inserted into the sheath, and
the treatment is performed on the treatment target site with the inserted treatment tool.

7. The treatment method according to claim 1, wherein
an auxiliary insertion tool for holding the treatment member is brought close to an opening, and
the treatment member is inserted into the maxillary sinus.

8. The treatment method according to claim 1, wherein
an auxiliary insertion tool for holding the endoscope is brought close to an opening, and
the endoscope is inserted into the maxillary sinus.

9. The treatment method according to claim 1, wherein
the treatment member is inserted into the maxillary sinus through the first opening after the first opening is formed.

10. The treatment method according to claim 1, wherein
the endoscope is inserted into the maxillary sinus through the second opening after the second opening is formed.
